(19) United States
(12) Patent Application Publication
(10) Pub. No.: US 2013/0345262 A1
(43) Pub. Date: Dec. 26, 2013

(54) METHOD FOR PREVENTION OF STROKE

(71) Applicants: Klaus DUGI, Dresden (DE); Andreas CLEMENS, Wiesbaden (DE); Thorsten LEHR, Ulm (DE); Herbert NOACK, Mannheim (DE)

(72) Inventors: Klaus DUGI, Dresden (DE); Andreas CLEMENS, Wiesbaden (DE); Thorsten LEHR, Ulm (DE); Herbert NOACK, Mannheim (DE)

(73) Assignee: BOEHRINGER INGELHEIM INTERNATIONAL GMBH, Ingelheim am Rhein (DE)

(21) Appl. No.: 13/923,762

(22) Filed: Jun. 21, 2013

(30) Foreign Application Priority Data
Jun. 25, 2012 (EP) 12173398.4

(51) Int. Cl. A61K 31/4439

(52) U.S. Cl. CPC A61K 31/4439
USPC 514/338

(57) ABSTRACT

A method for administering Dabigatran, optionally in the form of a pharmaceutically acceptable salt thereof, to a patient in need of an anticoagulant and/or antithrombotic medicament wherein the daily dose is determined by the dosage regimen including one measurement of dabigatran plasma level.

### FIELD OF THE INVENTION

The present invention relates to methods administering dabigatran etexilate, optionally in the form of a pharmaceutically acceptable salt thereof, that provide advantages over conventional warfarin and other vitamin K antagonist therapies.

### BACKGROUND TO THE INVENTION

Atrial fibrillation (AF) is a common cardiac arrhythmia which increases the risk of stroke, other embolic events, and death. AF affects 2.2 million people in the United States, and 4.5 million in the EU. AF is the most common heart rhythm disorder and is a major risk factor for stroke. The incidence of AF increases with age and nearly 6% of individuals over the age of 65 are affected. Patients with AF are at risk of developing clots due to the rapid irregular beating of the heart. AF increases the chance of stroke five-fold. As the consequences of stroke can be devastating, a primary aim of therapy is to decrease the risk of arterial thrombus formation and thromboembolism. Long-term anticoagulation therapy with vitamin K antagonists (VKAs or coumadins) such as warfarin is recommended for individuals with AF who are considered at moderate to high risk of stroke. These stroke, thrombosis, or embolism risk factors include age over 65 years, a history of a previous stroke or transient ischemic attack, hypertension, diabetes, or heart failure. Further risk factors for stroke are known to the physician and also defined hereinbelow.

VKAs, such as warfarin, reduce the risk of stroke by about 64% compared to placebo, but increase the risk of hemorrhage. Hart R G, Pearce L A, and Aguilar M I, Meta-analysis: Antithrombotic therapy to prevent stroke in patients who have nonvalvular atrial fibrillation, Ann of Intern Med., 2007, 146:857-867. When compared to placebo, warfarin also reduces mortality. Therefore, warfarin is recommended for patients with atrial fibrillation at risk for stroke. Fuster V, et al., ACC/AHA/ESC 2006 guidelines for the management of patients with atrial fibrillation—executive summary: a report of the American College of Cardiology/American Heart Association Task Force on Practice Guidelines and the European Society of Cardiology Committee for Practice Guidelines (Writing Committee to Revise the 2001 Guidelines for the Management of patients Patient with Arial Fibrillation), J Am Coll Cardiol, 2006, 48:854-906,

VKAs, such as warfarin, are cumbersome to use due to multiple diet and drug interactions and require frequent laboratory monitoring to adapt for the appropriate dosing. Therefore they are often not used, and discontinuation rates are high. Birman-Deych E, Radford M J, Nilasena D S, Gage B F, Use and Effectiveness of Warfarin in Medicare Beneficiaries with Atrial Fibrillation, Stroke, 2006, 37:1070-1074; Hylek E M, Evans-Molina C, Shea C, Henault L E, Regan S, Major Hemorrhage and Tolerability of Warfarin in the First Year of Therapy Among Elderly Patients with Atrial Fibrillation, Circulation, 2007, 115:2689-2696. Furthermore, even when on warfarin, many patients have inadequate anticoagulation. Connolly S J, Pogue J, Eikelboom J, Flaker G, Commerford P, Franzosi M G, Healey J S, Yusuf S, ACTIVE W Investigators. Benefit of oral anticoagulant over antiplatelet therapy in atrial fibrillation depends on the quality of international normalized ratio control achieved by centers and countries as measured by time in therapeutic range, Circulation, 2008, 118(20):2029-37. Accordingly, although warfarin reduces stroke in atrial fibrillation, it increases hemorrhage and is difficult to use. Thus, although anticoagulation therapy with warfarin has been shown to significantly reduce the incidence of stroke, only half of eligible patients are estimated to receive appropriate treatment due to a variety of barriers in administration and use of VKAs. Therefore, there is a need for new effective, safe, predictive and convenient anticoagulants.

Dabigatran etexilate is a compound of Formula (I)

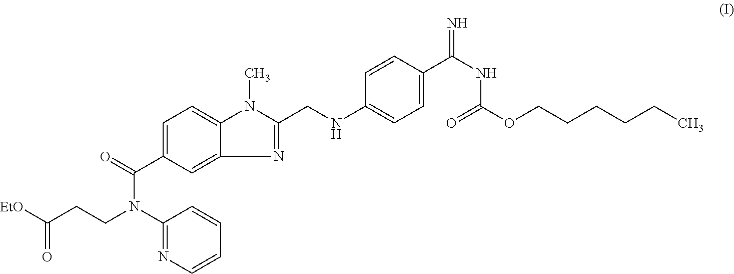 [SMILES: CCCCCCOC(=O)NC(=N)c1ccc(NCc2nc3cc(C(=O)N(CCC(=O)OCC)c4ccccn4)ccc3n2C)cc1]

is an oral direct thrombin inhibitor useful in the prophylaxis of thromboembolism in patients undergoing total knee or hip replacement and also suitable for the prevention of stroke, in particular in patients with atrial fibrillation. Other indications also exist, see, e.g., U.S. Patent Application Pub. Nos. 2008/0015176; 2008/0039391; and 2008/0200514. The compound of Formula (I) is already known from WO 98/37075 (corresponding to U.S. Pat. Nos. 6,087,380; 6,469,039; 6,414,008; and 6,710,055), in which compounds with a thrombin-inhibiting and thrombin time-prolonging activity are disclosed, under the name 1-methyl-2-[N-[4-(N-n-hexyloxycarbonylamidino)phenyl]aminomethyl]benzimidazol-5-ylcarboxylic acid-N-(2-pyridyl)-N-(2-ethoxycarbonylethyl)amides. Dabigatran etexilate is a double prodrug of dabigatran, the compound of Formula (II)

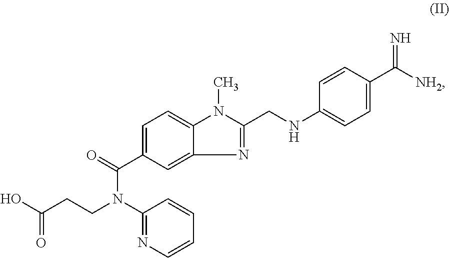 [SMILES: Cn1c(CNc2ccc(C(=N)N)cc2)nc2cc(C(=O)N(CCC(=O)O)c3ccccn3)ccc21]

i.e., dabigatran etexilate is only converted into the compound which is actually effective, namely dabigatran, in the body. Dabigatran etexilate is preferably administered in the form of its methanesulfonate salt, although also the salts of dabigatran etexilate with other pharmaceutically acceptable acids are encompassed in the context of the present invention. See, e.g., U.S. Patent Application Pub. No. 2006/0183779.

Dabigatran is a new oral direct thrombin inhibitor which has advantages over warfarin and other VKAs.

WO2010055021 describes a method comprising administering an effective amount of dabigatran etexilate for preventing stroke in a patient suffering from atrial fibrillation with an improved safety profile. The patient is at a reduced risk for an adverse bleeding event particularly when compared to conventional warfarin therapy. Concomitant the method provides a therapeutic efficacy in terms of stroke prevention which is equal to conventional warfarin therapy.

WO2010055022 describes a method comprising administering an effective amount of dabigatran etexilate for preventing stroke in a patient suffering from atrial fibrillation with an improved therapeutic efficacy and a good safety profile. The method provides an improved therapeutic effacy in terms of stroke prevention particularly when compared to conventional warfarin therapy. Concomittant the method provides a safety profile with a bleeding risk comparable with conventional warfarin therapy.

The aim of the present invention is to provide an optimal dosage i.e. dosage regimen for dabigatran etexilate, which meets the need of effective, safe, and convenient anticoagulant.

It is a further objective of the present invention to identify such a treatment or dosage regimen, which is optimized in terms of therapeutical efficacy and safety for patients of different age, gender, weight and physical constitution.

It is a further objective of the present invention to identify an optimal dosage regimen in terms of therapeutal efficacy and safety for dabigatran etexilate resulting in improved ischemic stroke prevention in conjunction with significant reduction of major bleeding events in AF (atrial fibrillation) patients, particularly when compared to treatment with warfarin.

It is a further objective of the present invention to identify an optimal dabigatran etexilate dosage regimen for the prevention of stroke in an AF patient, which provides an improved stroke prevention that is superior to warfarin treatment and in addition provides a reduced risk for an adverse bleeding event compared to warfarin treatment. Accordingly the advantages of the inventions described in WO2010055021 and WO2010055022 as outlined above are aimed at becoming a combined effect of the current invention.

All of the patents, patent applications, and documents cited herein are each hereby incorporated by reference in their entireties.

### BRIEF DESCRIPTION OF THE DRAWINGS

FIG. 1: Impact of dose adjustment based on dabigatran plasma level on major bleeds and on ischemic stroke/SEE compared to reference (150 mg b.i.d.) calculated as R=% Change Major Bleeds+2x % Change Ischemic Stroke/SEE. % Change is calculated as % change compared to 150 mg b.i.d treatment

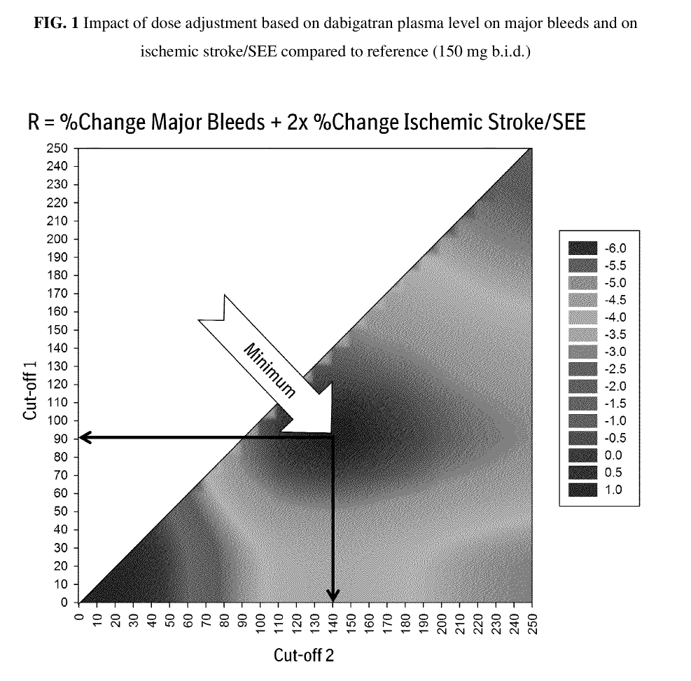

The values on the X and Y axis indicate the cut-off plasma levels leading to dose adjustment:

If Ctrough,ss<Cut-Off-1 then 150 mg b.i.d.
If Ctrough,ss>=Cut-off-1 and <Cut-off-2 then 110 mg b.i.d.
If Ctrough,ss>=Cut-off-2 then 75 mg b.i.d.

### DESCRIPTION OF THE INVENTION

Surprisingly it has been found that a dabigatran plasma concentration of 40 ng/mL to 110 ng/mL, preferably 49 ng/mL to 102 ng/mL, particularly preferred 80 ng/mL to 90 ng/mL, provides an optimal therapeutal efficacy, e.g. an optimum of stroke prevention, in conjunction with a significantly reduced major bleeding risk compared to warfarin treatment or compared to standard dabigatran treatment as outlined in WO2010055021 and WO2010055022.

The present invention solves the problems stated above.

As the exposure to dabigatran correlates with efficacy and also safety the present invention concentrates on the aspect that through plasma level measurements for the identification of the most appropriate (e.g. dabigatran plasma level or anticoagulant activity [e.g. measured by a anticoagulation test in the blood] which provides an optimal benefit/risk ratio) after a short exposure to a defined dose of dabigatran could serve as a better measure compared to patients characteristic over all (as e.g. mentioned in some labels of dabigatran). This becomes plausible as the patient characteristics affects in part (e.g. renal impairement) the through plasma levels of dabigatran. In achieving with this method a more precise predectibility and also lower variability of achieved plasma levels, e.-g. 40 ng/ml to 110 ng/ml, preferably 49 ng/ml to 102 ng/ml, particularly preferred 80 to 90 ng/ml, compared to the measured once in the RE-LY trial could optimize the efficacy and safety profile of dabigatran in comparison to warfarin. This would then be evaluated in patients of different age, gender, weight and physical constitution and provides with the measure of the trough plasma levels a decision for a more precise and optimized dose selection.

Accordingly the current invention relates to dabigatran etexilate, optionally in the form of a pharmaceutically acceptable salt thereof, for use as an anticoagulant and/or antithrombotic treatment in a patient in need thereof,

wherein the patient receives a specific dosage regimen determined according to steps A to C, wherein
step A comprises administering n mg b.i.d. to the patient for a specified period, wherein
   n denotes 50 to 300 mg b.i.d., preferably 50 to 220 mg b.i.d. and particularly preferred 75, 110 or 150 mg b.i.d.,
step B comprises measurement, optionally single measurement or multiple measurement, of the plasma level of dabigatran or the anticoagulant activity after the period as defined in step A, and
step C comprises adjusting/maintaining the daily dose
   to 75 mg b.i.d. following a plasma level of ≧(cut-off-2×n/150) ng/ml measured in step B or
   to 110 mg b.i.d. following a plasma level of ≧(cut-off-1×n/150) ng/ml and <(cut-off-2×n/150) ng/ml measured in step B
   or
   to ≧150 mg b.i.d. following a plasma level of <(cut-off-1×n/150) ng/ml measured in step B,
   wherein
   cut-off-1 denotes a plasma level measured according to step B from 70 to 130 ng/mL and
   cut-off-2 denotes a plasma level measured according to step B from 100 to 190 ng/mL.

Preferably the current invention relates to dabigatran etexilate, optionally in the form of a pharmaceutically acceptable salt thereof, wherein the dosage regimen is determined according to steps A to C,

wherein
step A comprises administering n mg b.i.d. to the patient for a specified period, wherein n denotes 50 to 300 mg b.i.d.,
step B comprises measurement, optionally single measurement or multiple measurement, of the plasma level of dabigatran or the anticoagulant activity after the period as defined in step A, and
step C comprises adjusting/maintaining the daily dose
to 75 mg b.i.d. following a plasma level of ≧(140×n/150) ng/ml measured in step B or
to 110 mg b.i.d. following a plasma level of ≧(90×n/150) ng/ml and <(140×n/150) ng/ml measured in step B or
to ≧150 mg b.i.d. following a plasma level of <(90×n/150) ng/ml measured in step B.

Also preferably the current invention relates to dabigatran etexilate, optionally in the form of a pharmaceutically acceptable salt thereof, wherein the dosage regimen is determined according to steps A to C,

wherein
step A comprises administering 150 mg b.i.d. to the patient for a specified period,
step B comprises measurement, optionally single measurement or multiple measurement, of the plasma level or the anticoagulant activity of dabigatran after the period as defined in step A, and
step C comprises adjusting/maintaining the daily dose
to 75 mg b.i.d. following a plasma level of ≧140 ng/ml measured in step B or
to 110 mg b.i.d. following a plasma level of ≧90 ng/ml and <140 ng/ml measured in step B or
to ≧150 mg b.i.d. following a plasma level of <90 ng/ml measured in step B.

Also preferably the invention relates to dabigatran etexilate, optionally in the form of a pharmaceutically acceptable salt thereof, wherein the patient in need of anticoagulant and/or antithrombotic treatment is suffering from atrial fibrillation.

Particularly preferred the invention relates to dabigatran etexilate as described above, optionally in the form of a pharmaceutically acceptable salt thereof, wherein the patient is selected from the group consisting of:
(a) having an age of at least 75 years;
(b) having a history of stroke;
I having a history of a transient ischemic attack;
(d) having a history of a thromboembolic event;
(e) having left ventricular dysfunction;
(f) having an age of at least 65 years and having high blood pressure;
(g) having an age of at least 65 years and having diabetes;
(h) having an age of at least 65 years and having coronary artery disease; and
(i) having an age of at least 65 years and having peripheral artery disease.
(j) having renal impairment

Also preferred in the present invention is dabigatran etexilate, optionally in the form of a pharmaceutically acceptable salt thereof, for preventing stroke, thrombosis or embolism.

Also preferred in the present invention is dabigatran etexilate, optionally in the form of a pharmaceutically acceptable salt thereof, wherein the patient in need of anticoagulant and/or antithrombotic treatment is a patient with a mechanical heart valve.

Also preferred in the present invention is dabigatran etexilate, optionally in the form of a pharmaceutically acceptable salt thereof, for preventing VTE after orthopaedic surgery.

Also preferred in the present invention is dabigatran etexilate, optionally in the form of a pharmaceutically acceptable salt thereof, wherein the patient has no risk factor for major bleeding events.

Particularly preferred in the present invention is dabigatran etexilate, optionally in the form of a pharmaceutically acceptable salt thereof, wherein the patient has at least one risk factor for major bleeding events.

Also particularly preferred in the present invention is dabigatran etexilate, optionally in the form of a pharmaceutically acceptable salt thereof, wherein the patient in need of an anticoagulant and/or antithrombotic treatment is a patient of 75 years or older.

Also particularly preferred in the present invention is dabigatran etexilate, optionally in the form of a pharmaceutically acceptable salt thereof, wherein the patient in need of an anticoagulant and/or antithrombotic treatment is a patient with renal impairment.

Also particularly preferred in the present invention is dabigatran etexilate, optionally in the form of a pharmaceutically acceptable salt thereof, wherein the risk factor for major bleeding events includes a history of earlier bleeding events.

Also particularly preferred in the present invention is dabigatran etexilate, optionally in the form of a pharmaceutically acceptable salt thereof, wherein the risk factor for major bleeding events includes a reduced creatinine clearance less than 90 mL/min, most preferably less than 80 mL/min.

Moreover preferred in the present invention is dabigatran etexilate, optionally in the form of a pharmaceutically acceptable salt thereof, wherein the specified period of step A requires at least 3 days.

The current invention relates to a method for administering dabigatran etexilate, optionally in the form of a pharmaceutically acceptable salt thereof, to a patient in need of an anticoagulant and/or antithrombotic medicament

characterized in that the daily dose is determined by the dosage regimen under steps A to C, wherein
step A comprises administering n mg b.i.d. to the patient for a specified period, wherein
   n denotes 50 to 300 mg b.i.d., preferably 50 to 220 mg b.i.d. and particularly preferred 75, 110 or 150 mg b.i.d.,
step B comprises measurement, optionally single measurement or multiple measurement, of the plasma level of dabigatran after the period as defined in step A, and
step C comprises adjusting/maintaining the daily dose
   to 75 mg b.i.d. following a plasma level of ≧(cut-off-2×n/150) ng/ml measured in step B or
   to 110 mg b.i.d. following a plasma level of ≧(cut-off-1×n/150) ng/ml and <(cut-off-2×n/150) ng/ml measured in step B
   or
   to ≧150 mg b.i.d. following a plasma level of <(cut-off-1×n/150) ng/ml measured in step B,
   wherein
   cut-off-1 denotes a plasma level measured according to step B from 70 to 130 ng/mL and
   cut-off-2 denotes a plasma level measured according to step B from 100 to 190 ng/mL.

Preferably cut-off-1 denotes 80 to 100 ng/ml, particularly preferred 90 ng/ml.

Preferably cut-off-2 denotes 120 to 170 ng/ml, particularly preferred 140 ng/ml.

Preferably the current invention relates to a method for administering dabigatran etexilate, optionally in the form of a pharmaceutically acceptable salt thereof, to a patient in need of an anticoagulant and/or antithrombotic medicament

characterized in that the daily dose is determined by the dosage regimen under steps A to C, wherein
step A comprises administering n mg b.i.d. to the patient for a specified period, wherein
   n denotes 50 to 300 mg b.i.d., preferably 50 to 220 mg b.i.d. and particularly preferred 75, 110 or 150 mg b.i.d.,
step B comprises measurement, optionally single measurement or multiple measurement of the plasma level of dabigatran after the period as defined in step A, and
step C comprises adjusting/maintaining the daily dose
   to 75 mg b.i.d. following a plasma level of ≧(140×n/150) ng/ml measured in step B or
   to 110 mg b.i.d. following a plasma level of ≧(90×n/150) ng/ml and <(140×n/150) ng/ml measured in step B
   or
   to ≧150 mg b.i.d. following a plasma level of <(90×n/150) ng/ml measured in step B.

Also preferably the current invention relates to a method for administering dabigatran etexilate, optionally in the form of a pharmaceutically acceptable salt thereof, to a patient in need of an anticoagulant and/or antithrombotic medicament

characterized in that the daily dose is determined by the dosage regimen under steps A to C, wherein
step A comprises administering 150 mg b.i.d. to the patient for a specified period,
step B comprises measurement, optionally single measurement or multiple measurement, of the plasma level of dabigatran after the period as defined in step A, and
step C comprises adjusting/maintaining the daily dose
to 75 mg b.i.d. following a plasma level of ≧140 ng/ml measured in step B or
to 110 mg b.i.d. following a plasma level of ≧90 ng/ml and <140 ng/ml measured in step B or
to ≧150 mg b.i.d. following a plasma level of <90 ng/ml measured in step B.

Also preferably the invention relates to a method, wherein the patient in need of an anticoagulant and/or antithrombotic medicament is suffering from atrial fibrillation.

Particularly preferred the invention relates to any of the methods described above, wherein the patient is selected from the group consisting of:
(a) having an age of at least 75 years;
(b) having a history of stroke;
I having a history of a transient ischemic attack;
(d) having a history of a thromboembolic event;
(e) having left ventricular dysfunction;
(f) having an age of at least 65 years and having high blood pressure;
(g) having an age of at least 65 years and having diabetes;
(h) having an age of at least 65 years and having coronary artery disease; and
(i) having an age of at least 65 years and having peripheral artery disease.
(j) having renal impairment

Also preferred in the present invention are any of the methods described above for preventing stroke, thrombosis or embolism, particularly preferred for preventing stroke in AF patients (SPAF).

Also preferred in the present invention are any of the methods described above wherein the patient in need of an anticoagulant and/or antithrombotic medicament is a patient with a mechanical heart valve.

Also preferred in the present invention are any of the methods described above for preventing VTE after orthopaedic surgery, particularly preferred for preventing VTE after total knee or hip arthroplasty.

Also preferred in the present invention are any of the methods described above wherein the patient has no risk factor for major bleeding events.

Particularly preferred in the present invention are any of the methods described above, wherein the patient has at least one risk factor for major bleeding events.

Also particularly preferred in the present invention are any of the methods described above, wherein the patient in need of an anticoagulant and/or antithrombotic medicament is a patient of 75 years or older, preferably 80 years or older.

Also particularly preferred in the present invention are any of the methods described above, wherein the patient in need of an anticoagulant and/or antithrombotic medicament is a patient with renal impairment.

Also particularly preferred in the present invention are any of the methods described above, wherein the risk factor for major bleeding events includes a history of earlier bleeding events.

Also particularly preferred in the present invention are any of the methods described above, wherein the risk factor for major bleeding events includes a reduced creatinine clearance less than 80 mL/min, preferably less than 50 mL/min, most preferably less than 30 mL/min

Moreover preferred in the present invention are any of the methods described above, wherein the specified period of step A requires at least 3 days, preferably 3 to 10, more preferably 5 to 7 days.

In particular the current invention enables the physician to decide on the appropriate medication for a patient that suffered from AF without a risk factor for major bleeding events or at least one risk factor for major bleeding events as defined herein below.

The method according to the invention focuses on the prevention of thrombosis, embolism, or stroke, preferably stroke, particularly in patients without a risk factor for major bleeding events but also in patients that are characterized by risk factors for major bleeding events. One important risk factor for major bleeding events is the age of at least 75 years. Another risk factor for major bleeding events may include a history of earlier bleeding events and the like. Furthermore, a reduced creatinine clearance (Cockcroft-Gold method or equivalent) less than 90 mL/min, preferably less than 80 mL/min, preferably less than 50 mL/min, most preferably less than 30 mL/min, could possibly amount to a risk factor for major bleeding events. Further risk factors for major bleeding events are known to the physician and also defined hereinbelow.

Treatment of these patients at risk for major bleeding events is particularly useful as the patient is at a reduced risk for a major bleeding event when compared to treatment with warfarin.

AF is a chronic condition, which is presently not curable but can only be relieved. Patients suffering from AF require to be treated with dabigatran etexilate lifelong. Thus, there is a need for determining a dosage regimen suitable for long-term treatment using dabigatran etexilate for patients suffering from AF. Specifically, there exists a need for determining a dosage range and treatment scheme (posology), which balances thromboembolic prevention and minimizes risk factors, especially bleeding, in particular in patients with an identified risk factor for major bleeding events. In the treatment of AF, the suitability of a patient having risk factors, e.g., stroke and bleeding, is determined by a skilled physician.

The dabigatran etexilate, optionally in the form of a pharmaceutically acceptable salt thereof-containing pharmaceutical compositions of the invention will be delivered for a time sufficient to achieve the desired physiological effect, i.e., prevention or treatment of thrombosis. Typically, the pharmaceutical compositions will be delivered as an oral composition twice a day. The compositions may be administered for a defined time or indefinitely.

When administered in accordance with the methods of the invention, dabigatran etexilate, optionally in the form of a pharmaceutically acceptable salt thereof, provides the patient with a safe and therapeutically efficacious method for the prevention or treatment of thrombosis. The dabigatran etexilate, optionally in the form of a pharmaceutically acceptable salt thereof, is able to prevent thrombosis but not result in an adverse bleeding event.

Dabigatran can be made into pharmaceutical formulations, see, e.g., U.S. Patent Application Pub. No. 2005/0038077; U.S. Patent Application Pub. Nos. 2005/0095293; 2005/0107438; 2006/0183779; and 2008/0069873. In addition, dabigatran can be administered with other active ingredients, see, e.g., U.S. Patent Application Pub. Nos. 2006/0222640; 2009/0048173; and 2009/0075949.

In a preferred embodiment the dose unit is a solid form, such as a tablet, capsule, granulate, powder, and the like. For example, such formulations are presented in the Formulations section below. In a particular preferred embodiment the solid form is a capsule containing dabigatran etexilate, coated on isolated tartaric acid core pellets.

### DEFINITION OF TERMS AND CONVENTIONS USED

Terms not specifically defined herein should be given the meanings that would be given to them by one of skill in the art in light of the disclosure and the context. As used in the specification and appended claims, however, unless specified to the contrary, the following terms have the meaning indicated and the following conventions are adhered to.

The term “bleeding event” comprises “minor hemorrhage”, “minor bleeding event”, “major hemorrhage”, “major bleeding event”, “major bleeds”, “life-threatening bleeding” and “life-threatening bleeding event”.

The terms “minor hemorrhage” and “minor bleeding event” means a bleeding event that does not fulfill the criteria for a major bleeding event.

The terms “major hemorrhage”, “major bleeding event”, and “major bleeds” mean a reduction in hemoglobin level of at least 2.0 g/L or transfusion of at least 2 units of blood or symptomatic bleeding in a critical area or organ.

The terms “life-threatening bleeding” and “life-threatening bleeding event” mean a subset of major bleeding event that includes fatal bleeding, symptomatic intracranial bleeding, bleeding with hemoglobin decrease of more than 5.0 g/L, or requiring transfusion of more than 4 units of blood or requiring inotropic agents or necessitating surgery.

The term “warfarin” means an anticoagulant that acts by inhibiting vitamin K-dependent coagulation factors and is sold under the brand names Coumadin, Jantoven, Marevan, and Waran. Chemically, it is 3-(α-acetonylbenzyl)4-hydroxycoumarin and is a racemic mixture of the R- and S-enantiomers. Warfarin is a synthetic derivative of coumarin, a chemical found naturally in many plants. Warfarin decreases blood coagulation by inhibiting vitamin K epoxide reductase, an enzyme that recycles oxidized vitamin K to its reduced form.

The term “conventional warfarin therapy” relates to the amount of warfarin administered to a patient according to the ACC/AHA/ESC Practice Guidelines (Fuster et al., JACC, Vol. 48, No. 4, Aug. 15, 2006, 854-906; see, e.g., page 859, Class 1 recommendation, points 3 and 4), incorporated herein by reference. The RELY Clinical Trial used conventional warfarin therapy as the comparator.

The term “dabigatran etexilate” means a compound of Formula (I) including its pharmaceutically acceptable salts. The single dosage amount of dabigatran etexilate in any salt form in mg refers to the free base, i.e., to the free base of Formula (I). The dose amount of prodrug dabigatran etexilate is based on the weight of its free base.

The term “dabigatran” is the compound of Formula (II) in its free base form.

The term “AF” means atrial fibrillation, a cardiac arrhythmia.

The term “SPAF” means stroke prevention in atrial fibrillation.

The term “non-valvular atrial fibrillation” means AF in the absence of rheumatic mitral stenosis or a prosthetic heart valve.

The terms “thrombotic events” and “thromboembolic events” mean an occurrence of thromboembolies or stroke. “Thrombosis” is the formation of a blood clot (thrombus) inside a blood vessel, obstructing the flow of blood through the circulatory system. If a clot breaks free, an embolus is formed. “Thromboembolism” is the formation in the blood vessel of a clot that breaks loose and is carried by the blood stream to plug another vessel. The clot may plug a vessel in the lungs (pulmonary embolism), brain (stroke), gastrointestinal tract, kidneys, or leg.

The terms “non-CNS systemic embolism” or “SE” means that a piece of blood clot that breaks off from a clot, often in the left atrial chamber of the heart, flows through the systemic circulation and blocks a pat of the circulation other than the brain (when it blocks brain circulation it's a stroke).

The term stroke comprises “hemorrhagic stroke” and “ischemic stroke”.

The term “hemorrhagic stroke” means a bleed inside the brain.

The terms “subarachnoid hemorrhage” or “subarachnoid bleed” mean a bleeding into the subarachnoid space, the area between the arachnoid membrane and the pia mater surrounding the brain.

The terms “subdural hemorrhage” or “subdural bleed” mean a bleeding within the inner meningeal layer of the dura, the outer protective covering of the brain, surrounding the brain.

The term “intracranial hemorrhage” or “ICH” means a hemorrhagic stroke including subdural bleed plus subarachnoid bleed. Hemorrhagic stroke is bleed inside the brain and subdural hemorrhage and subarachnoid hemorrhage are on the surface of the brain but outside the brain and ICH is a composite of these different bleeds.

The term “stroke, thrombosis, or embolism risk factors” means the risk factors that are known to statistically increase the risk of thrombosis, embolism, or stroke. These risk factors include: AF, having a history of stroke; having a history of a transient ischemic attack; having a history of a thromboembolic event; having left ventricular dysfunction; having an age of at least 65 years and having high blood pressure; having an age of at least 65 years and having diabetes; having an age of at least 65 years and having coronary artery disease; and, having an age of at least 65 years and having peripheral artery disease. Accordingly, generally stroke, thrombosis, or embolism risk factors include age; heredity; gender; prior stroke, transient ischemic attack, or heart attack; high blood pressure; cigarette smoking; diabetes mellitus; carotid or other artery disease; atrial fibrillation or other heart disease; sickle cell disease; high blood cholesterol; diets high in saturated fat, trans fat, cholesterol, and sodium; and physical inactivity and obesity.

The National Stroke Association (US) indicates that one is at a “high risk of stroke” if they have at least 3 of the following risk factors: a blood pressure at 140/90 or higher; a cholesterol level of 240 or higher; has diabetes; is a smoker; suffers from atrial fibrillation; is overweight; does not exercise; or, has a history of stroke in their family.

The National Stroke Association (US) indicates that one is at a “moderate risk of stroke” if they have 4-6 of the following: a blood pressure of 120-139/80-89; a cholesterol level of 200-239; is borderline for diabetes; is trying to quit smoking; is not aware of having an irregular heartbeat; is slightly overweight; exercises sometimes; and is not sure of a family history of stroke.

The National Stroke Association (US) indicates that one is at a “low risk of stroke” if they have 6-8 of the following: a blood pressure of 120/80 or lower; a cholesterol of 200 or lower; does not have diabetes; is not a smoker; does not have an irregular heartbeat; is at a healthy weight; exercises regularly; and does not have a history of stroke in their family.

The term “risk factors for major bleeding events” means various risk factors that are known to statistically increase the risk of a patient having a major bleeding event. Risk factors for major bleeding events are known to the physician working in the field. For safety reasons, the existence of risk factors for major bleeding events need to be determined by the physician in every patient. As an example, the risk factors for major bleeding events can be grouped into demographics (age, gender, and nursing facility residence). As an example, patients being at the age of 75 years or greater could be considered a risk factor for major bleeds. These risk factors can also include alcohol/drug abuse, concomitant diseases (anemia, cancer, stroke, transient ischemic attacks, MI, hypertension, heart failure/cardiomyopathy, ischemic heart disease, diabetes, hepatic failure, or peptic ulcer disease) and concomitant risks for injury (risk for falls, cognitive impairment, or surgery during index hospitalization). Risk factors for major bleeding events are also present in patients having a history of earlier bleeding events or in patients having a reduced creatinine clearance, for instance, less than 80 mL/min, less than 50 mL/min, or less than 30 mL/min

The term “b.i.d.” means that the daily dosage is administered in two separate administrations, which are timely separated by at least 4 hours, preferably at least 6 hours and more preferably at least 8 hours. Consequently, a dosage of 110 mg b.i.d. means a daily dosage of 220 mg, which is administered twice daily at a single dose of 110 mg.

The dosages referred to herein are based on the amount of dabigatran etexilate free base (i.e., the compound depicted in Formula (I)). If dabigatran etexilate is administered in form of one of its pharmaceutically acceptable salts the amount of the salt that is used is to be calculated from the indicated dosage. As an example, if dabigatran etexilate is administered in form of its methanesulfonate salt a dosage of 110 mg equals an amount of 126.83 mg of dabigatran etexilate methanesulfonate.

The term “pharmaceutically acceptable salt” means a salt of a compound of the invention which is, within the scope of sound medical judgment, suitable for use in contact with the tissues of humans and lower animals without undue toxicity, irritation, allergic response, and the like, commensurate with a reasonable benefit/risk ratio, generally water or oil-soluble or dispersible, and effective for their intended use. The term includes pharmaceutically-acceptable acid addition salts and pharmaceutically-acceptable base addition salts. As the compounds of the present invention are useful in both free base and salt form, in practice, the use of the salt form amounts to use of the base form. Lists of suitable salts are found in, e.g., S. M. Birge et al., J. Pharm. Sci., 1977, 66, pp. 1-19, which is hereby incorporated by reference in its entirety. Most preferred according to the invention is the methanesulfonic acid addition salt of dabigatran etexilate which is also referred to herein as dabigatran etexilate methanesulfonate.

The term “prevent” means to keep from happening or continuing and relates to a statistical reduction in the risk of an event occurring. “Preventing” is synonymous with “reducing the risk” or “demonstrating a lower incidence” of an event occurring. Reducing the risk or demonstrating a lower incidence means that there is a statistical reduction or lowering in occurrence of the event by at least 1% or greater. Preferably, this reduction is by 7% or greater, 10% or greater, 20% or greater, 26% or greater, 34% or greater, 50% or greater, 64% or greater and 74% or greater. These reductions include confidence intervals greater than 50%, greater than 75%, greater than 80%, greater than 90%, greater than 95%, greater than 98% and greater than 99%. Confidence intervals of greater than 95% are preferred.

The terms “plasma level” or “plasma level concentration” mean trough levels at steady state (C_{trough,ss}) following administration of 50 to 300 mg b.i.d., preferably 50 to 220 mg b.i.d., preferably 150 mg b.i.d., 110 mg b.i.d. or 75 mg b.i.d. dabigatan etexilate, more preferably 150 mg b.i.d. Preferably C_{trough,ss} is measured 10 to 16 hours, more preferably 12 hours, following the last administration.

Basically values of corresponding plasma peak concentration levels are deducible from plasma concentration trough levels (C_{trough,ss}) and vice versa applying formula (ii)

Ctrough,ss [ng/mL]=C2h,ss [ng/mL]*0.47182+12.06 (ii)

wherein

C2h,ss denotes mean peak levels at steady state (C2h,ss) following administration of 50 to 300 mg b.i.d., preferably 50 to 220 mg b.i.d., preferably 150 mg b.i.d., 110 mg b.i.d. or 75 mg b.i.d. dabigatan etexilate, more preferably 150 mg b.i.d. Preferably C2h,ss is measured 1 to 3 hours, more preferably 2 hours, following the last administration.

Hence the methods described above may also be applied when C2h,ss levels in consideration of formula (ii) are measured.

The methods of the invention provide a safe and therapeutically effective amount of dabigatran etexilate, optionally in the form of a pharmaceutically acceptable salt thereof. By “safe and therapeutically effective amount” is intended an amount of dabigatran etexilate, optionally in the form of a pharmaceutically acceptable salt thereof, that when administered in accordance with the invention is free from major complications, such as an adverse bleeding event, that cannot be medically managed, and that provides for objective improvement in patients by preventing or treating thrombosis. It is considered and managed by the present invention that the therapeutically effective amount may vary from patient to patient depending upon age, weight, severity of symptoms, general health, physical condition, and the like.

### Determination of Dabigatran Plasma Levels

Although clinical monitoring of dabigatran is generally not required, a reliable laboratory method to measure the exposure of dabigatran is needed for the methods of the invention.

One such method is described in WO10086329, wherein the method comprises the quantitative determination of dabigatran in blood samples.

### Formulations

Dabigatran can be made into pharmaceutical formulations, see, e.g., U.S. Patent Application Pub. Nos. 2005/0038077; 2005/0095293; 2005/0107438; 2006/0183779; and 2008/0069873. In addition, dabigatran can be administered with other active ingredients, see, e.g., U.S. Patent Application Pub. Nos. 2006/0222640; 2009/0048173; and 2009/0075949. A pharmaceutically acceptable carrier or diluent that is conventionally used in the art can be used to facilitate the storage, administration, and/or the desired effect of the therapeutic ingredients. A suitable carrier should be stable, i.e., incapable of reacting with other ingredients in the formulation. Such carriers are generally known in the art. A thorough discussion of formulation and selection of pharmaceutically acceptable carriers, stabilizers, and the like can be found in Remington's Pharmaceutical Sciences (18^{th} ed.; Mack Pub. Co.: Eaton, Pa., 1990), herein incorporated by reference.

Dabigatran etexilate is preferably formulated as the methanesulfonate salt (WO 03/074056). Examples illustrating dosage forms according to the present invention and methods for the production thereof are disclosed in WO2009118322, WO2009/118321 and WO2010/007016

### Combinations

It is further recognized that the dabigatran etexilate or pharmaceutically acceptable salt thereof may be co-administered with an antiplatelet agent. Antiplatelet agents include cyclooxygenase inhibitors such as aspirin; adenosine diphosphate (ADP) receptor inhibitors; phosphodiesterase inhibitors; glycoprotein IIB/IIIA inhibitors; adenosine reuptake inhibitors; and the like. In one embodiment, the antiplatelet agent is aspirin and is administered at less than or equal to 100 mg per day.

The following example is offered by way of illustration and not by way of limitation.

### Experimental

Exposure-response relationship was intensively characterized in >8000 AF patients from RE-LY showing a significant relationship between dabigatran trough concentrations at steady-state (Ctrough,ss) and major bleeding events and ischemic stroke/SEE (systemic embolism events) events.

Extensive and comprehensive clinical trial simulation analyses were performed to investigate the impact of dabigatran dose titration on the outcome in AF patients.

Cut-off values between 0 ng/mL and 250 ng/mL (step size 10 ng/mL) were evaluated to identify optimal dabigatran steady state plasma levels for dose adjustment, based on patients who received a dose of 150 mg b.i.d dabigatran etexilate methansulfonate.

For each combination 500 clinical trials with 5000 patients (randomly selected from RELY study) each were simulated and the dabigatran exposure, ischemic stroke/SEE (systemic embolism events) and major bleeding event rates were projected.

The benefit of dose adjustment based on plasma levels compared to a reference treatment (dabigatran etexilate 150 mg bid) is calculated according to formula (i):

R=% Change Major Bleeds+2x % Change Ischemic Stroke/SEE (i)

wherein R represents a risk value indicating the weighted risk of major bleeds and ischemic stroke or SEE.

Dabigatran Ctrough,ss values of 90 ng/mL and 140 ng/mL were identified as promising cut-off values to assign dabigatran doses of 150 mg bid.

Compared to a reference treatment (dabigatran etexilate 150 mg bid), dose adjustment showed a significant reduction of major bleeding events (RR 0.8) while the ischemic stroke protection was maintained (RR 1.06).

Compared to warfarin treatment, dose adjustment showed a significant reduction of ischemic stroke/SEE (RR 0.8) and major bleeding events (RR 0.6) (see Table 1).

For example RR=0.8 means dose adjustment according to the current invention achieved a reduced risk from 100% to 80% of ischemic stroke/SEE compared to warfarin treatment.

TABLE 1
Effect of dabigatan etexilate dose adjustment
compared to Warfarin treatment
Cut-off Ctrough, ss values of 90 ng/mL and 140 ng/mL; n = 150 mg b.i.d.

| | Rel. | |
|---|---|---|
| Dose Adjustment vs. Warfarin | Risk | 95% CI |
| Ischemic Stroke/SEE | 0.80 | (0.58-1.11) |
| Major Bleeding | 0.60 | (0.50-0.72) |

CI means Confidence Interval

We claim:

1. A method for anticoagulant and/or antithrombotic treatment in a patient in need thereof, comprising the dosing regimen steps of:
(a) administering n mg b.i.d. dabigatran etexilate, optionally in the form of a pharmaceutically acceptable salt thereof, to the patient for a specified period, wherein n denotes 50 mg to 300 mg;
(b) measuring once or multiple times a plasma level of dabigatran after the period specified in step (a); and
(c) adjusting and maintaining a daily dose to
(i) 75 mg b.i.d. following a plasma level of ≧(cut-off-2×n/150) ng/mL measured in step (b); or
(ii) 110 mg b.i.d. following a plasma level of ≧(cut-off-1×n/150) ng/mL and <(cut-off-2×n/150) ng/mL measured in step (b); or
(iii) ≧150 mg b.i.d. following a plasma level of <(cut-off-1×n/150) ng/mL measured in step (b),
wherein cut-off-1 denotes a plasma level measured according to step (b) from 70 to 130 ng/mL and cut-off-2 denotes a plasma level measured according to step (b) from 100 to 190 ng/mL.

2. The method according to claim 1, wherein the n in step (a) denotes 50 mg to 220 mg b.i.d.

3. The method according to claim 1, wherein the n in step (a) denotes 75, 110 or 150 mg b.i.d.

4. The method according to claim 1, wherein adjusting and maintaining the daily dose in step (c) is further defined to
(i) 75 mg b.i.d. following a plasma level of ≧(140×n/150) ng/mL measured in step (b); or
(ii) 110 mg b.i.d. following a plasma level of ≧(90×n/150) ng/mL and <(140×n/150) ng/mL measured in step (b); or
(iii) ≧150 mg b.i.d. following a plasma level of <(90×n/150) ng/mL measured in step (b).

5. The method according to claim 1, wherein step (a) further comprises administering 150 mg b.i.d. to the patient for a specified period and step (c) the daily dose is adjusted and maintained to
(i) 75 mg b.i.d. following a plasma level of ≧140 ng/mL measured in step (b); or
(ii) 110 mg b.i.d. following a plasma level of ≧90 ng/mL and <140 ng/mL measured in step (b); or
(iii) ≧150 mg b.i.d. following a plasma level of <90 ng/mL measured in step (b).

6. The method according to claim 1, wherein the patient in need of the anticoagulant and/or antithrombotic treatment is suffering from atrial fibrillation.

7. The method according to claim 1, wherein the patient is selected from the group consisting of:
(a) having an age of at least 75 years;
(b) having a history of stroke;
(c) having a history of a transient ischemic attack;
(d) having a history of a thromboembolic event;
(e) having left ventricular dysfunction;
(f) having an age of at least 65 years and having high blood pressure;
(g) having an age of at least 65 years and having diabetes;
(h) having an age of at least 65 years and having coronary artery disease;
(i) having an age of at least 65 years and having peripheral artery disease; and
(J) having renal impairment.

8. The method according to claim 1, wherein the anticoagulant and/or antithrombotic treatment is for preventing stroke, thrombosis or embolism.

9. The method according to claim 1, wherein the patient in need of an anticoagulant and/or antithrombotic treatment is a patient with a mechanical heart valve.

10. The method according to claim 1, wherein the anticoagulant and/or antithrombotic treatment is for preventing VTE after orthopaedic surgery.

11. The method according to claim 1, wherein the patient in need of an anticoagulant and/or antithrombotic treatment is a patient that has no risk factor for major bleeding events.

12. The method according to claim 1, wherein the patient in need of an anticoagulant and/or antithrombotic treatment is a patient that has at least one risk factor for major bleeding events.

13. The method according to claim 1, wherein the patient in need of an anticoagulant and/or antithrombotic treatment is a patient of 75 years or older.

14. The method according to claim 1, wherein the patient in need of an anticoagulant and/or antithrombotic treatment is a patient with renal impairment.

15. The method according to claim 12, wherein the risk factor for major bleeding events includes a history of earlier bleeding events.

16. The method according to claim 12, wherein the risk factor for major bleeding events includes a reduced creatinine clearance less than 80 mL/min.

17. The method according to claim 1, wherein the specified period of step (a) requires at least 3 days.
